(19) United States
(12) Patent Application Publication
(10) Pub. No.: US 2026/0232558 A1
(43) Pub. Date: Aug. 13, 2026

(54) SUNSCREEN COMPOSITION WITH NON-NANO ORGANIC UV FILTER

(71) Applicant: BASF SE, Ludwigshafen am Rhein (DE)

(72) Inventor: Ullrich MENGE, Grenzach-Wyhlen (DE)

(21) Appl. No.: 19/159,645

(22) PCT Filed: Feb. 2, 2024
(86) PCT No.: PCT/EP2024/052589

§ 371 Date: Aug. 26, 2025

(30) Foreign Application Priority Data
Feb. 27, 2023 (EP) 23158728.8

(51) Int. Cl. A61K 8/49; A61K 8/44; A61Q 17/04

(52) U.S. Cl. CPC A61K 8/496; A61K 8/445; A61K 8/4966; A61Q 17/04; A61K 2800/30; A61K 2800/412

(57) ABSTRACT

The invention relates to a sunscreen or daily care composition comprising i) at least one particulate, micronized organic UV filter having a particle size D_{N}50 of more than 100 nm and a particle size Dv90 of less than 20 μm and ii) at least one further inorganic or organic UV filter.

### FIELD OF THE INVENTION

The present invention relates to a sunscreen or daily care composition comprising i) at least one micronized organic UV filter and ii) at least one further inorganic or organic UV filter.

### BACKGROUND OF THE INVENTION

The use of sunscreen compositions to protect the skin from the damaging effects of sun irradiation is becoming more and more ubiquitous. At the same time, the expectations of consumers and regulators regarding safety, performance, sustainability and cost of sunscreen and daily care compositions are constantly increasing. Consequently, there is a need to develop high-performing sunscreen compositions that simultaneously fulfil the other requirements from consumers and regulators.

Sunscreen compositions comprise UV filters that absorb and may further scatter UV irradiation effectively. One group of UV filters that has been found to be particularly effective in absorbing and scattering UV irradiation is the group of particulate, nano-sized insoluble organic crystalline UV filters. Particulate, nano-sized insoluble organic UV filters overcome the common solubility issues of organic UV filters by circumventing the problem of low solubility altogether and providing the UV filter as aqueous suspension that can be used in different types of sunscreen products. Additionally, particulate, nano-sized UV filters have been found to be particularly effective UV absorbents. Due to the nano-sized scale of the UV filter particles, the number of molecules that are hit by UV light is greater in a nano-sized particulate material than in a material with larger particles for the same mass of material. In addition, the surface coverage of the skin is increased for the same mass of material using a nano-sized particles versus larger particles. These effects have been described in detail in the art, for example in B. Herzog et al., “Physical properties of organic particulate UV absorbers used in sunscreens II. UV-attenuating efficiency as function of particle size”, Journal of Colloid and Interface Science 276 (2004), 354-363 and in S. Mueller et al., “Micronization as a Tool in the Development of Innovative UV Filters”, SÖFW-Journal, 131 (2005), 32-38.

Methods to prepare such particulate, nano-sized insoluble organic UV filters have been described in the art, for example in WO 97/03643 A1 and WO 2009/003934 A1. The particulate, nano-sized insoluble organic UV filters are obtained by micronization in a ball mill using grinding beads.

The nano-sized UV filters disclosed in the art display excellent UV absorbance properties. However, nano-sized materials have recently come under attack due to customer concerns regarding safety, in particular regarding possible skin permeation of nano-sized materials.

Although these concerns may be scientifically unfounded, there is a growing demand for nano-free sunscreen and daily care products from consumers.

### Objective of the Invention

Against the above-outlined background it was an object of the present invention to provide a sunscreen or daily-care composition with excellent UV absorbance properties that does not rely on nano-sized UV filters.

### SUMMARY OF THE INVENTION

It was surprisingly found that a sunscreen or daily care composition comprising
i) at least one particulate, micronized organic UV filter having a particle size D_{N}50 of more than 100 nm and a particle size Dv90 of less than 20 μm and
ii) at least one further inorganic or organic UV filter
exhibits excellent UV absorbance properties without relying on nano-sized organic UV filters.

### Definitions

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art. As used herein the term “does not comprise” or “free of” means in the context that the composition of the present invention is free of a specific compound or group of compounds, which may be combined under a collective term, that the composition does not comprise said compound or group of compounds in an amount of more than 0.8% by weight, based on the total weight of the composition. Furthermore, it is preferred that the composition according to the present invention does not comprise said compounds or group of compounds in an amount of more than 0.5% by weight, preferably the composition does not comprise said compounds or group of compounds at all.

When referring to compositions and the weight percent of the therein comprised ingredients it is to be understood that according to the present invention the overall amount of ingredients does not exceed 100% (±1% due to rounding).

The term “sunscreen composition” or “sunscreen” refers to any topical product, which absorbs and which may further reflect and scatter certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic and inorganic UV filters, as well as other ingredients or additives, e.g., emulsifiers, emollients, viscosity regulators, stabilizers, preservatives, or fragrances.

The term “daily care composition” refers to any topical product. A daily care composition which contains UV filters absorbs and may further reflect and scatter certain parts of UV radiation and is used as an everyday care product for the human body, e.g. for face or body. The daily care composition may comprise one or more active agents, e.g., organic and/or inorganic UV filters, as well as other ingredients or additives, e.g., emulsifiers, emollients, viscosity regulators, stabilizers, preservatives, or fragrances. Suitable daily care composition are according to the present invention, e.g. leave-on face and body care products.

Suitable leave-on products for face and body are, e.g. daily care preparation, sunscreen compositions, decorative preparations, and skin care preparations.

Suitable decorative preparations are, e.g., lipsticks, nail varnishes, eye shadows, mascaras, dry and moist make-up, rouge, powders, and depilatory agents.

Suitable skin care preparations are e.g., moisturizing, refining, and lifting preparations. The cited daily care compositions can be in the form of creams, ointments, pastes, foams, gels, lotions, powders, make-ups, sprays, sticks or aerosols. The daily-care compositions with UV protection comprise the UV filters i) and ii) and protect against damages on human skin by UV light.

The term “UV filter” or “ultraviolet filter” as used herein refers to organic or inorganic compounds, which can absorb and may further reflect and scatter UV radiation caused by sunlight. UV-filter can be classified based on their UV protection curve as UV-A, UV-B, or broadband filters. Preferably, the term “UV filter” comprises or consists of any UV filter as defined in the Annex VI (version of 03.12.2020) of the Regulation (EC) No 1223/2009 of the European Parliament and of the Council.

Water soluble UV filters have a solubility in water of at least 2% by weight, preferably at least 3% by weight, more preferably at least 5% by weight at 25° C.

Oil soluble UV filters have a solubility in oils (emollients) of at least 2% by weight, preferably at least 3% by weight, more preferably at least 5% by weight at 25° C.

The prefix Cn-Cm indicates in each case the possible number of carbon atoms in the group.

The term “C₁₂-C₁₅ alkyl benzoate” refers to esters of benzoic acid with fatty alcohols containing a C₁₂-C₁₅-alkyl chain. C₁₂-C₁₅ alkyl chain is defined as an alkyl chain with C₁₂, C₁₃, C₁₄ or C₁₅ chain length.

The term “Cn-Cm carboxylic acids” as used herein denotes in each case a linear or branched carboxylic acids having from n to m carbon atoms, such as 6 to 24 carbon atoms.

The term “Cn-Cm alcohols” as used herein denotes in each case a linear or branched alcohol having from n to m carbon atoms, such as having from 3 to 24 carbon atoms, or from 6 to 24 carbon atoms, or from 1 to 22 carbon atoms.

The term “C2-C12 dicarboxylic acids” as used herein denotes in each case a dicarboxylic acid having from 2 to 12 carbon atoms such as butanedioic acid (succinic acid), pentanedioic acid (glutaric acid), hexanedioic acid (adipic acid), or decanedioic acid (sebacic acid).

The term “dialkyl ether” as used herein denotes in each case a linear or branched dialkyl ether having a total of from 12 to 36 carbon atoms and comprising at least one ether moiety.

The term “C6-C22 alcohol carbonates” as used herein denotes in each case a linear or branched alcohol carbonates having 6 to 22 carbon atoms and comprising at least one functional group consisting of a carbonyl group flanked by two alkoxy groups.

The term “alkyl” as used herein denotes in each case a straight-chain or branched alkyl group having exemplarily from 1 to 18 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “alkoxy” as used herein denotes in each case a linear or branched alkyl group which is bonded via an oxygen atom and has usually from 1 to 20 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “carboxyalkyl” as used herein includes carboxymethyl, carboxyethyl, carboxypropyl, carboxyisopropyl, carboxybutyl, carboxyisobutyl, carboxyamyl, carboxyhexyl, carboxyheptyl, carboxyoctyl, carboxyisooctyl, carboxynonyl, carboxydecyl, carboxyundecyl, carboxydodecyl, carboxytetradecyl, carboxyhexadecyl, and carboxyoctadecyl, carboxymethyl being preferred.

The term “cycloalkyl” as used herein denotes in each case a monocyclic cycloaliphatic radical having usually from 3 to 10 or from 5 to 8 carbon atoms, such as cyclopropyl, cyclobutyl, cyclopentyl, cyclohexyl, cycloheptyl, cyclooctyl, cyclononyl and cyclodecyl or cyclopropyl, cyclobutyl, cyclopentyl and cyclohexyl.

The term “substituted” as used herein, means that a hydrogen atom bonded to a designated atom is replaced with a specified substituent, provided that the substitution results in a stable or chemically feasible compound. Unless otherwise indicated, a substituted atom may have one or more substituents and each substituent is independently selected.

The term “emollient” relates to cosmetic specific oils used for protecting, moisturizing and lubricating the skin. The word emollient is derived from the Latin word mollire, to soften. In general, emollients prevent evaporation of water from the skin by forming an occlusive coating. Alternatively, or additionally, the emollients improve the distribution of the product on the skin. Emollients can be divided into different groups depending on their polarity index.

The term “sensitive skin” refers to skin of which the natural barrier function is weakened and has broken due to a trigger. A trigger can be for example cold weather, extremely hot water and critical ingredients, which may be included in sunscreen or daily care compositions.

The term “sun protection factor (SPF)” as used herein indicates how well the skin is protected by a sunscreen composition mainly from UV-B radiation. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimeter (mg/cm²).

The definition of “broadband” protection (also referred to as broad-spectrum or broad protection) may be based on the “critical wavelength” or on UVA-PF. For broadband coverage, UV-B and UV-A protection must be provided. According to the US requirements, a critical wavelength of at least 370 nm is required for achieving broad spectrum protection. Furthermore, it is recommended by the European Commission that all sunscreen or cosmetic compositions should have an UV-A protection factor, which is at least one third of the labelled sun protection factor (SPF), e.g. if the sunscreen composition has an SPF of 30 the UVA protection factor has to be at least 10.

The term “critical wavelength” is defined as the wavelength at which the area under the UV protection curve (% protection versus wavelength) represents 90% of the total area under the curve in the UV region (290-400 nm). For example, a critical wavelength of 370 nm indicates that the protection of the sunscreen composition is not limited to the wavelengths of UV-B, i.e. wavelengths from 290-320 nm, but extends to 370 nm in such a way that 90% of the total area under the protective curve in the UV region are reached at minimum 370 nm.

The term “administration” refers to the application of a sunscreen or daily care composition to the skin of a person.

The term “at least one particulate, micronized organic UV filter i)” refers to particles of the UV filter. It is understood that the at least one particulate, micronized organic UV filter refers to one type of UV filter, not a single particle of the UV filter. Consequently, the at least one particulate, micronized organic UV filter i) refers to a plurality of particles of the at least one UV filter with a specific size distribution of the particles.

As used herein the term “nano” or “nano material” follows the recommendation of the European Commission 2011/696/EU. Accordingly, in a nano material 50% or more of particles, based on a number-based size distribution, are smaller than 100 nm, including constituent particles in aggregates or agglomerates. In the spirit of this invention a non-nano particulate UV filter i) contains less than 50% and more preferably less than 30% of particles smaller than 100 nm, referring to a number-based particle size distribution.

The size of the UV filter particles in the present invention is defined by the particle size distribution of a set of particles.

The particle size distribution may be characterized with respect to particle volume (mass), or to the number of particles. For instance, Dv90=1 μm means that 90% of the volume (or mass) of the dispersed material consists of particles smaller than 1 μm, and 10% are larger. In contrast, D_{N}50=100 nm refers to the number-based size distribution and indicates that 50% of all particles in the sample are smaller than 100 nm. As a rule of the thumb, in a polydisperse sample the small particles dominate number-based distributions D_{N} while large particles dominate Dv. Measurement techniques differ in their sensitivity towards particle number or particle mass. Volume based distributions may be obtained by laser diffraction, and number-based distributions by electron microscopy. As for laser diffraction, many commercial instruments are available, for example from Anton Paar (PSA series), Microtrac MRB (Sync) or Malvern Panalytical (Mastersizer series). Depending on sensitivity and resolution of the selected instrument, the numerical results characterizing the particle size distribution will differ within some minor range. The skilled person knows how to deal with these deviations, and such instruments are routinely used in R&D and quality control labs. In contrast, electron microscopy is more expensive and less frequently used.

This distinction between D_{N} and Dv is relevant for the “nano” classification of materials. In accordance with the recommendation of the European Commission 2011/696/EU, a micronized dispersion with a D_{N}50 value of 100 nm or below is a nano material. The methods found in the literature for the production of micronized organic UV absorbers end up in nano dispersions (WO2018069200). This seems desirable as the efficiency of the UV absorber dispersion increases with decreasing particle size. Furthermore, depending on the mechanical properties of the UV absorber crystals, the breaking mechanism in a grinding device may generate nano material out of crystals that are still in the μm range.

The term “dispersion” as used herein refers to a system in which distributed particles of one material are dispersed in a continuous phase of another material. The two phases may be in the same or different states of matter. A specific subtype of a dispersion is the “suspension”, wherein solid parts are dispersed (i.e. not dissolved) in a fluid.

### PARTICULAR EMBODIMENTS OF THE INVENTION

The present invention relates to a sunscreen or daily care composition comprising
i) at least one particulate, micronized organic UV filter having a particle size D_{N}50 of more than 100 nm and a particle size Dv90 of less than 20 μm and
ii) at least one further inorganic or organic UV filter.

It was found that a sunscreen or daily care composition with these components allows for a surprisingly high sun protection without the need of nano-sized particulate UV filters. This finding stands in contrast to the established concept that nano-sized particulate UV filters exhibit a particularly high sun protection that cannot be achieved by non-nano sized particulate UV filters of the same type. Previous studies had found that nano-sized particulate UV filters such as MBBT greatly outperformed the same UV filters in non-nano size. Surprisingly and in contrast to this previous understanding regarding nano-sized UV filters, sunscreen products which comprise the components i) and ii) in accordance with the invention exhibit similar sun protection compared to the same sunscreen products with component i) in nano-size. This was a completely unexpected finding in view of the previously established theoretical understanding of particulate UV filters. Without being bound to any specific scientific theory, it is believed that in a sunscreen or daily care product, the complex interaction of the different components, in particular of the different UV filters i) and ii), results in a different UV extinction behavior than the analysis of aqueous suspension consisting only of particulate, micronized UV filters as UV absorbers suggested.

The present invention allows for the provision of sunscreen or daily care products that provide the same level of protection to consumers without the need of nano-sized UV particulate filters. This is a major breakthrough in view of consumer demands regarding cosmetics which do not contain nano-sized ingredients and increasing requirements by regulatory bodies regarding nano material comprising cosmetic products.

### Particulate UV Filter i)

The at least one particulate, micronized organic UV filter i) has a particle size D_{N}50 of more than 100 nm and a particle size Dv90 of less than 20 μm. This particle size range was found to be suited for optimal UV extinction. Larger particles do not provide the excellent UV extinction desired for sunscreen and daily care products and are therefore not as well suited for these applications. Furthermore, larger particles may result in an uncomfortable haptic sensation and whitening appearance on the skin. Smaller particles may be considered or close to being considered nano materials and could therefore come under increased scrutiny by consumers and regulators.

As previously stated, D_{N} and D_{V} values allow to describe the size distribution of the particles of the at least one particulate, micronized organic UV filter. The D_{N} value describes the number of particles in a set of particles that is below a certain threshold. For example, it is to be understood that an organic UV filter having a particle size D_{N}50 of more than 100 nm may also be expressed as an organic UV filter, wherein less than 50% of particles (number evaluation) have a particle size of 100 nm or less. D_{N} values are particularly useful to identify and quantify the smaller particles within a set of particles of a certain material. The particle size D_{N}50 may be determined by e.g. transmission electron microscopy (TEM) or scanning electron microscopy (SEM), preferably by transmission electron microscopy (TEM).

In the present invention, the at least one particulate, micronized organic UV filter has a particle size D_{N}50 of more than 100 nm. Consequently, less than 50% of the particles of the at least one particulate, micronized organic UV filter i) have a particle size below 100 nm.

In a preferred embodiment of the invention, the at least one particulate, micronized organic UV filter i) has a particle size D_{N}10, preferably determined by transmission electron microscopy, of 30 nm or more, preferably of 35 nm to 400 nm, more preferably of 50 nm to 200 nm. In this manner, the number of very small particles of the UV filter i) is particularly small.

In another preferred embodiment of the invention, the at least one particulate, micronized organic UV filter i) has a particle size D_{N}50, preferably determined by transmission electron microscopy, of 120 nm or more, preferably of from 0.12 to 0.5 μm, and more preferably from 0.12 to 0.3 μm. According to this preferred embodiment, less than 50% of the particles of component i) have a particle size below 120 nm.

In another preferred embodiment of the invention, the at least one particulate, micronized organic UV filter i) has a particle size D_{N}90, preferably determined by transmission electron microscopy, of 200 nm or more, preferably of from 0.2 to 1.0 μm, more preferably from 0.2 to 0.5 μm.

The particle size distribution of the particles of the particulate, micronized organic UV filter i) can also be described by the Dv particle size distribution. The Dv value refers to the volume (or mass) distribution of particle sizes instead of the number of particle sizes. Dv values are particularly useful to describe larger particles within a set of particles of a certain material.

The Dv90 value of the at least one particulate, micronized organic UV filter i) is less than 20 μm. That means that 90% of the volume (or mass) of the particles have a particle size of less than 20 μm. Larger Dv90 values than 20 μm may result in an unpleasant skin feel and a whitening effect. Preferably, the at least one particulate, micronized organic UV filter i) has a particle size Dv90 of less than 10 μm, more preferred of less than 5 μm, even more preferred of less than 3 μm, and most preferred of less than 2.2 μm.

According to another preferred embodiment of the invention, the at least one particulate, micronized organic UV filter i) has a particle size Dv50 of less than 0.8 μm, preferably less than 0.6 μm, more preferred less than 0.5 μm and most preferred less than 0.4 μm.

According to another preferred embodiment of the invention, the at least one particulate, micronized organic UV filter i) has a Dv10 value of more than 0.1 μm, preferably of from 0.1 to 0.4 μm.

The Dv90, Dv50 and Dv10 values are preferably determined by laser diffraction using a “Mastersizer 2000” from Malvern Instruments Ltd., UK.

It is directly apparent that the different D_{N} and Dv values describe the particle size distribution of the at least one particulate, micronized organic UV filter in different ways. Consequently, different ways of describing the particle size distribution in form of different Dv and D_{N} values can be combined variably to describe the particle size distribution of the particles.

In a preferred embodiment of the present invention, the at least one particulate, micronized organic UV filter i) is an insoluble organic UV filter. In this connection it is to be understood that the term insoluble UV filter refers to UV filters that are not soluble in water and cosmetic oils at 25° C. To the contrary, water-soluble UV filters have a solubility in water of at least 2% by weight, preferably at least 3% by weight, more preferably at least 5% by weight at 25° C. Oil soluble UV filters have a solubility in oils (emollients) of at least 2% by weight, preferably at least 3% by weight, more preferably at least 5% by weight at 25° C. The insoluble organic UV filter i) according to this preferred embodiment do not fulfil either of these two requirements and are therefore considered “insoluble” in water and oil at 25° C.

It is particularly preferred that the at least one micronized organic UV filter i) is selected from the group consisting of tris-biphenyl triazine, 1,1′-(1,4-piperazinediyl)bis[1-[2-[4-(diethylamino)-2-hydroxybenzoyl]phenyl]-methanone, phenylene bis-diphenyltriazine, and 2,2′-methylenebis[6-(2H-1,2,3-benzotriazol-2-yl)-4-(2,4,4-trimethylpentan-2-yl)phenol]. These four UV filters are the most common particulate organic UV filters in the art and are particularly well suited as component i) in the inventive sunscreen or daily care composition.

Tris-biphenyl triazine has the following structure:

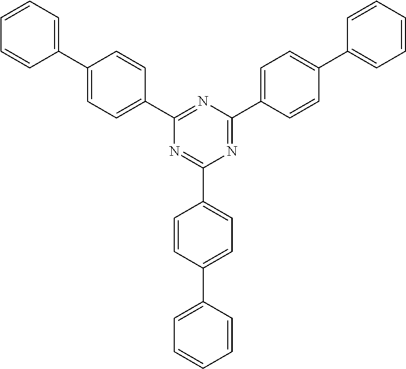 [SMILES: c1ccc(-c2ccc(-c3nc(-c4ccc(-c5ccccc5)cc4)nc(-c4ccc(-c5ccccc5)cc4)n3)cc2)cc1]

1,1′-(1,4-piperazinediyl)bis[1-[2-[4-(diethylamino)-2-hydroxybenzoyl]phenyl]-methanone has the following structure:

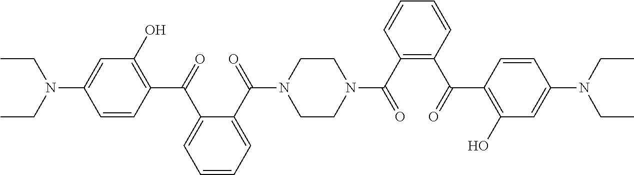 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

Phenylene bis-diphenyltriazine has the following structure:

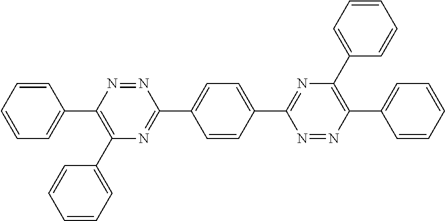 [SMILES: c1ccc(-c2nnc(-c3ccc(-c4nnc(-c5ccccc5)c(-c5ccccc5)n4)cc3)nc2-c2ccccc2)cc1]

2,2′-methylenebis[6-(2H-1,2,3-benzotriazol-2-yl)-4-(2,4,4-trimethylpentan-2-yl)phenol] has the following structure:

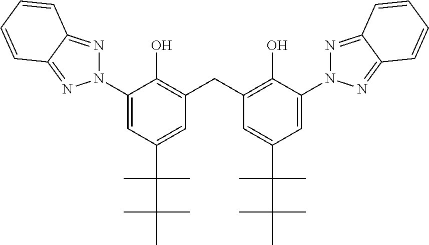 [SMILES: CC(C)(C)C(C)(C)c1cc(Cc2cc(C(C)(C)C(C)(C)C)cc(-n3nc4ccccc4n3)c2O)c(O)c(-n2nc3ccccc3n2)c1]

It is more preferred that the at least one micronized organic UV filter i) is selected from the group consisting of tris-biphenyl triazine, 1,1′-(1,4-piperazinediyl)bis[1-[2-[4-(diethylamino)-2-hydroxybenzoyl]phenyl]-methanone, and 2,2′-methylenebis[6-(2H-1,2,3-benzotriazol-2-yl)-4-(2,4,4-trimethylpentan-2-yl)phenol].

According to one embodiment of the invention, the at least one micronized organic UV filter i) is 1,1′-(1,4-piperazinediyl)bis[1-[2-[4-(diethylamino)-2-hydroxybenzoyl]phenyl]-methanone.

According to another embodiment of the invention, the at least one micronized organic UV filter i) is tris-biphenyl triazine. Tris-biphenyl triazine (also: TBPT) is a broad-spectrum UV filter that as a high performance over the entire UV-A and UV-B spectrum.

According to another embodiment of the invention, the at least one micronized organic UV filter i) is 2,2′-methylenebis[6-(2H-1,2,3-benzotriazol-2-yl)-4-(2,4,4-trimethylpentan-2-yl)phenol]. 2,2′-methylenebis[6-(2H-1,2,3-benzotriazol-2-yl)-4-(2,4,4-trimethylpentan-2-yl)phenol] is a highly effective broadband UV filter which covers the entire UV-A and UV-B spectrum. It is the most preferred UV filter i) according to the present invention.

According to a preferred embodiment of the invention, the inventive sunscreen or daily care composition comprises the at least one particulate, micronized organic UV filter i) in an amount of from 0.1 to 20 wt. %, in particular from 0.5 to 15 wt. %, preferably from 1 to 10 wt. %, more preferred from 1.5 to 8 wt. %, and most preferred from 2 to 5 wt. %, each based on the total weight of the sunscreen or daily care composition.

The at least one micronized organic UV filter i) having a particle size D_{N}50 of more than 100 nm and a particle size Dv90 of less than 20 μm can be prepared with the method disclosed in patent application PCT/EP2022/072536 which is herewith incorporated by reference. The micronization process to prepare the particulate, micronized organic UV filter i) according to PCT/EP2022/072536 comprises the step of milling a suspension of at least one organic UV filter in a mixture of water and a hydrophobic additive in a milling apparatus at a temperature of 35 to 90° C.

It is preferred that the at least one particulate, micronized organic UV filter i) is provided in form of an aqueous suspension. The micronization process to prepare the non-nano organic particulate UV filter i) used in the sunscreen or daily care composition of the present invention provides an aqueous suspension of the particulate UV filter in an aqueous medium as is apparent from the disclosure of PCT/EP2022/072536. It is preferred that the aqueous suspension is then used as such in the sunscreen or daily care composition.

The aqueous suspension preferably comprises a dispersing agent. The dispersing agent is relevant for the micronization process. Generally, any type of dispersing agent commonly employed for micronization processes for use in cosmetics can be used.

Suitable dispersing agents are in particular polyglycerol alkyl ester, preferably polyglycerol monoalkyl ester, and alkyl polyglucoside, preferably having the formula CnH2n+1O(C6H10O5)xH, in which n is an integer ranging from 8 to 16 and x is the mean polymerization level of the glucoside moiety (C6H10O) and ranges from 1.4 to 1.6, or an ester thereof. Further suitable dispersing agents are disclosed in WO2009068469. According to the present invention, the polyglycerol monoalkyl ester has preferably a mean degree of polymerization of glycerol of 5 or more.

In one preferred embodiment, the at least one polyglycerol monoalkyl ester is selected from the group consisting of decaglyceryl caprate, decaglyceryl monolaurate, decaglyceryl myristate, decaglyceryl oleate, decaglyceryl stearate, decaglyceryl isostearate, hexaglyceryl cap rate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, pentaglyceryl isostearate, and combinations thereof. In a particular preferred embodiment, the at least one polyglycerol monoalkyl ester is decaglyceryl monolaurate (INCI polyglyceryl-10 laurate). Polyglycerol monoalkyl esters having an HLB (hydrophilic-lipophilic balance) of 14.5 or more are preferable and having an HLB of 15 or more are more preferable. The HLP value is determined by the formula

$$HLB = 20 \cdot Mh / M ,$$

wherein Mh is the molecular mass of the hydrophilic portion of the molecule and M is the molecular mass of the whole molecule.

Polyglycerol monoalkyl esters having an HLB of less than 14.5 may take a longer time for dispersion of micronized methylene bis-benzotriazolyl tetramethylbutylphenol in water phase components. Examples of polyglycerol monoalkyl esters with a mean degree of polymerization of 5 or more and having an HLB of 14.5 or more may include decaglyceryl caprate, decaglyceryl monolaurate, decaglyceryl myristate, decaglyceryl oleate, decaglyceryl stearate, decaglyceryl isostearate, hexaglyceryl laurate, pentaglyceryl laurate, pentaglyceryl myristate, pentaglyceryl stearate, and pentaglyceryl oleate, and those having an HLB of 15 or more may include decaglyceryl caprate and decaglyceryl monolaurate.

Particularly preferred are polyglyceryl monolaurate, particularly decaglyceryl monolaurate, and decyl glucoside.

Preferably, the alkyl polyglucoside consists of a C1-C12ester of the compound of formula CnH2n+1O(C6H10O5)xH, namely an ester formed by reacting a C1-C12 carboxylic acid with one or more free OH group of the glucoside moiety (C6H10O). In this connection, it is preferred that the ester is formed by reacting formic, acetic, propionic, butyric, sulfosuccinic, citric, or tartaric acid, with one or more free OH groups on the glucoside moiety (C6H10O).

Preferably, the aqueous suspension comprises the dispersing agent in an amount of 1 to 20 wt. %, more preferably of 3 to 15 wt. % based on the total weight of the aqueous suspension.

According to another preferred embodiment of the invention, the aqueous suspension comprises the at least one particulate, micronized organic UV filter i) in an amount of 10 to 65 wt. %, in particular 30 to 60 wt. %, based on the total weight of the aqueous suspension. The weight ratio of the dispersing agent to the at least one particulate, micronized organic UV filter i) in the aqueous suspension is preferably from 0.01 to 0.5, more preferably from 0.05 to 0.4, and in particular from 0.07 to 0.3.

Further excipients may be added to the preparation, such as thickeners, anti-foaming agents, pH-adjusters, preservatives.

The aqueous suspension comprising the at least one particulate, micronized organic UV filter i) preferably also comprises a hydrophobic additive. Preferably, the hydrophobic additive is selected from the group consisting of alcohols having from 6 to 18 carbon atoms; C6-C24 carboxylic acids; esters of C6-C24 carboxylic acids with C3-C24 alcohols; esters of hydroxycarboxylic acids with C6-C24 alcohols; esters of carboxylic acids with polyhydric alcohols; liquid mono-/di-/tri-glyceride mixtures based on C6-C18 carboxylic acids; esters of C6-C24 alcohols with aromatic carboxylic acids or with oxo carboxylic acids; tricarboxylic acid esters; esters of C2-C12 dicarboxylic acids with alcohols having from 1 to 22 carbon atoms or polyols having from 2 to 10 carbon atoms and from 2 to 6 hydroxy groups; substituted cyclohexanes; C6-C22 alcohol carbonates; symmetric or asymmetric dialkyl ethers having a total of from 12 to 36 carbon atoms; ring-opening products of epoxidized carboxylic acid esters with polyols; silicone oils; aliphatic or naphthenic hydrocarbons; diol esters; and mixtures thereof. The hydrophobic additive has been found to be useful to enhance Ostwald-ripening of the UV filter particles in the micronization process, which leads to dissolution of nano particles formed in the grinding process and induces crystal growth which counteracts the grinding action in the mill. In this way, the optimal non-nano size distribution can be obtained in the grinding process.

In a preferred embodiment of the present invention, the hydrophobic additive is comprised in the aqueous suspension in an amount of 0.01 to 10.0 wt.-%, preferably of 0.01 to 5.0 wt-%, more preferably of 0.01 to 3.0 wt.-%, and in particular of 0.05 to 1.0 wt.-%, based on the total amount of the aqueous suspension.

### Additional UV Filter ii)

The sunscreen or daily care composition of the present invention further comprises at least one additional UV filter ii). The additional UV filter ii) may be an organic or an inorganic UV filter.

According to a preferred embodiment, the sunscreen or daily care composition comprises at least one further inorganic or organic UV filter ii) that is a UVA absorbing filter, a broad spectrum (UVA+UVB) filter or a UVB absorbing filter. The combination of at least one UVA absorbing filter, at least one broad spectrum filter and at least one UVB absorbing filter in the inventive sunscreen or daily care composition is a preferred embodiment.

According to a preferred embodiment, the sunscreen or daily care composition according to the invention is characterized in that the at least one further inorganic or organic UV filter ii) is selected from the group consisting of

### UVA Absorbing Filters:

4-(tert.-butyl)-4′-methoxydibenzoylmethane (INCI butyl methoxydibenzoylmethane),
hexyl 2-[4-(diethylamino)-2-hydroxybenzoyl]benzoate (INCI diethylamino hydroxybenzoyl hexyl benzoate),
[(3Z)-3-[[4-[(Z)-[7,7-dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (INCI terephthalylidene dicamphor sulfonic acid),
disodium phenyl dibenzimidazole tetrasulfonate;

### Broad Spectrum (UVA+UVB) Absorbing Filters:

2,4-bis-{[4-(2-ethyl-hexyloxy)-2-hydroxy]-phenyl}-6-(4-methoxyphenyl)-1,3,5 triazine (INCI bis-ethylhexyloxyphenyl methoxyphenyl triazine),
2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane),
Zinc oxide;

### UVB Absorbing Filters:

2-phenyl-1H-benzimidazole-5-sulfonic acid (INCI phenylbenzimidazole sulfonic acid),
4,4′,4″-(1,3,5-triazin-2,4,6-triyltriimino)tris-benzoic acid tris(2-ethylhexyl) ester (INCI ethylhexyl triazone),
(RS)-2-ethylhexyl-2-hydroxybenzoate (INCI ethylhexyl salicylate),
dimethicone diethyl benzalmalonate,
diethylhexyl butamido triazone,
2-ethoxyethyl(2Z)-2-cyano-2-[3-(3-methoxypropylamino)cyclohex-2-en-1-ylidene]acetate,
Titanium dioxide,
Cerium oxide;
   and mixtures thereof.

The at least one further organic or inorganic UV filter ii) is preferably selected from the group consisting of
4-(tert.-butyl)-4′-methoxydibenzoylmethane (INCI butyl methoxydibenzoylmethane),
hexyl 2-[4-(diethylamino)-2-hydroxybenzoyl]benzoate (INCI diethylamino hydroxybenzoyl hexyl benzoate),
2,4-bis-{[4-(2-ethyl-hexyloxy)-2-hydroxy]-phenyl}-6-(4-methoxyphenyl)-1,3,5 triazine (INCI bis-ethylhexyloxyphenyl methoxyphenyl triazine),
2-phenyl-1H-benzimidazole-5-sulfonic acid (INCI phenylbenzimidazole sulfonic acid),
[(3Z)-3-[[4-[(Z)-[7,7-dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (INCI terephthalylidene dicamphor sulfonic acid),
disodium phenyl dibenzimidazole tetrasulfonate,
(RS)-2-ethylhexyl-(2E)-3-(4-methoxyphenyl) prop-2-enoate (INCI ethylhexyl methoxycinnamate),
4,4′,4″-(1,3,5-triazin-2,4,6-triyltriimino)tris-benzoic acid tris(2-ethylhexyl) ester (INCI ethylhexyl triazone),
dimethicone diethyl benzalmalonate,
diethylhexyl butamido triazone,
2-ethoxyethyl(2Z)-2-cyano-2-[3-(3-methoxypropylamino)cyclohex-2-en-1-ylidene]acetate,
2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane),
Titanium dioxide,
Zinc oxide,
Cerium oxide,
   and mixtures thereof.

The combination of the at least one particulate, micronized organic UV filter i) with any one of these organic and inorganic UV filters provides a sunscreen composition with excellent sun protection properties.

It is further particularly preferred that the at least one further organic or inorganic UV filter ii) is an organic UV filter selected from the group consisting of
4-(tert.-butyl)-4′-methoxydibenzoylmethane (INCI butyl methoxydibenzoylmethane),
hexyl 2-[4-(diethylamino)-2-hydroxybenzoyl]benzoate (INCI diethylamino hydroxybenzoyl hexyl benzoate),
2,4-bis-{[4-(2-ethyl-hexyloxy)-2-hydroxy]-phenyl}-6-(4-methoxyphenyl)-1,3,5 triazine (INCI bis-ethylhexyloxyphenyl methoxyphenyl triazine),
4,4′,4″-(1,3,5-triazin-2,4,6-triyltriimino)tris-benzoic acid tris(2-ethylhexyl) ester (INCI ethylhexyl triazone),
2-phenyl-1H-benzimidazole-5-sulfonic acid (INCI phenylbenzimidazole sulfonic acid),
[(3Z)-3-[[4-[(Z)-[7,7-dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (INCI terephthalylidene dicamphor sulfonic acid), and mixtures thereof.

These are particularly useful UV filters that exhibit excellent sun protection properties while being environmentally benign and safe.

Most preferred, the at least one further organic or inorganic UV filter ii) is an organic UV filter selected from the group consisting of
hexyl 2-[4-(diethylamino)-2-hydroxybenzoyl]benzoate (INCI diethylamino hydroxybenzoyl hexyl benzoate),
4,4′,4″-(1,3,5-triazin-2,4,6-triyltriimino)tris-benzoic acid tris(2-ethylhexyl) ester (INCI ethylhexyl triazone), and mixtures thereof.

Diethylamino hydroxybenzoyl hexyl benzoate (DHHB) and 4,4′,4″-(1,3,5-triazin-2,4,6-triyltriimino)tris-benzoic acid tris(2-ethylhexyl) ester (INCI ethylhexyl triazone) (EHT) are highly effective UV filters which combined with the at least one particulate, micronized organic UV filter i) provide particularly potent sunscreen or daily care compositions over the entire relevant UV spectrum.

According to another preferred embodiment, the daily care or sunscreen composition is free of all of the UV-filters 2-ethylhexyl-2-cyano-3,3-diphenyl-2-propenoate (INCI octocrylene), (RS)-2-ethylhexyl-(2E)-3-(4-methoxyphenyl) prop-2-enoate (INCI ethylhexyl methoxycinnamate), and 3,3,5-trimethyl-cyclohexyl salicylate (INCI Homosalate). There is a growing concern that these UV filters may have negative health effects, either directly or through possible decomposition products deriving from these UV filters. Consequently, it is preferable that the inventive composition is free of these filters.

According to a preferred embodiment, the amount of the at least one further inorganic or organic UV filter ii) is from 0.1 to 20 wt. %, preferably from 0.5 to 15 wt. % and most preferred from 1 to 10 wt. %, each based on the total weight of the sunscreen or daily care composition.

According to a preferred embodiment of the invention, the at least one further organic or inorganic UV filter ii) is not a nano UV filter, i.e. the UV filter does not have a size distribution that can be considered as nano-material.

### Further Components of the Sunscreen or Daily Care Composition

The invention sunscreen or daily care composition preferably comprises additives, in particular selected from the group consisting of emulsifiers, emollients, viscosity regulators (thickeners), sensory enhancers, adjuvants, preservatives, perfumes and combinations thereof.

### Preferred Emulsifiers Include

glucose derivatives such as cetearyl glucoside, arachidyl glucoside, lauryl glucoside, polyglyceryl-3 methylglucose distearate, methyl glucose sesquistearate;
sucrose derivative such as sucrose polystearate, sucrose palmitate;
sorbitol derivatives;
glycerides of fatty acids such as glyceryl stearate, glyceryl oleate;
glumatic acid derivatives such as sodium stearoyl glutamate;
sulfosuccinic acid derivatives such as disodium cetearyl sulfosuccinate;
phosphoric acid derivatives such as potassium cetyl phosphate;
fatty acid esters of polyglyceryl such as polyglyceryl-3-diisostearate, polyglyceryl-2-dipolyhydroxystearate;
oxyalkenylated organomodified silicone/polysiloxane/polyalkyl/polyether copolymers and derivatives.

### Preferred Emollients Include

esters of linear or branched fatty acids with linear or branched fatty alcohols such as propylheptyl caprylate, coco caprylate, isopropyl myristate, ethylhexyl palmitate;
esters of aromatic carboxylic acids with linear or branched fatty alcohols such as C₁₂-C₁₅-alkyl benzoate, ethylhexyl benzoate, phenethyl benzoate;
dicarboxylic acid esters with linear or branched alcohols such as dibutyl adipate, dicaprylyl carbonate, diisopropyl sebacate;
esters of hydroxycarboxylic acids with linear or branched fatty alcohols;
esters of linear or branched fatty acids with polyhydric alcohol such as butylene glycol dicaprylate/dicaprate;
mono-, di-, tri-glycerides based on C₆-C₁₈ fatty acids such as caprylic/capric triglycerides, coco glycerides;
guerbet alcohols such as octyldodecynol;
hydrocarbons such as hydrogenated polyisobutene, mineral oil, squalene, isohexadecane;
ethers such as dicaprylyl ether;
silicone derivatives (organomodified polysiloxanes) such as dimethylpolysiloxane, cyclic silicones.

### Preferred Thickeners Include

fatty alcohols such as cetyl alcohol, cetearyl alcohol, stearyl alcohol;
fatty acids such as stearic acid;
fatty acid esters such as myristyl stearate;
waxes such as beeswax, carnauba wax, microcrystalline wax, ceresin, ozocerite;
polysaccharides or derivatives such as xanthan gum, guar gum, agar gum, alginates, gellan gum, carragheenan;
polyacrylates or homopolymers of reticulated acrylic acids or polyacrylamides such as carbomers, acrylate copolymers, acrylate/C10-C30-alkyl acrylate crosspolymer, acrylate/beheneth-25 methacrylate copolymer;
silicate derivatives such as magnesium silicates;
cellulose derivatives such as hydroxypropyl cellulose.

### Preferred Sensory Enhancers Include

polyamide derivatives such as nylon-12;
polymethyl methacrylates;
silica;
mica;
polymethylsilsesquioxane;
polyethylene;
starch derivatives such as aluminum starch octenylsuccinate;
dimethicone derivatives;
boron nitride;
HDI/trimethylol hexyllactone crosspolymer.

### Preferred Humectant Include

1,2 pentadiol;
1,2 hexandiol;
1,2 octandiol;
1,2 decandiol;
2-Methyl-1,3-propandiol.

### Preferred Adjuvants Include

tocopherol derivatives;
retinol derivatives;
ascorbic acid derivatives;
bisabolol;
allantoin;
panthenol;
chelating agents (EDTA, EDDS, EGTA, phytic acid, piroctone olamine);
ethylhexyl glycerin;
hydroxyacetophenone;
caprylhydroxymic acid;
propellants such as propane, butane, isobutene, dimethyl ether;
styrene/PVP or styrene acrylamide copolymers;
insect repellants such as butylacetylaminopropionate.

### Preferred Preservatives Include

benzyl alcohol;
zingerone.

Preferred perfumes are selected from the group consisting of limonene, citral, linalool, alpha-isomethylionon, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzylalcohol, benzylbenzoate, benzylcinnamate, benzylsalicylate, bergamot oil, bitter orange oil, butylphenylmethylpropioal, cardamom oil, cedrol, cinnamal, cinnamylalcohol, citronnellylmethylcrotonate, lemon oil, coumarin, diethylsuccinate, ethyllinalool, eugenol, Evernia furfuracea extract, Evernia prunastri extract, farensol, guajak wood oil, hexylcinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin and combinations thereof.

In connection with the above preferred embodiments, it is to be understood that if the sunscreen or daily care composition comprises two or more additives, combinations of the additives as defined above are also part of the invention.

In connection with the above preferred and particularly preferred embodiments, it is to be understood that the sunscreen or daily care composition may further comprise water. In case water is present in the sunscreen or daily care composition it is to be understood that it is preferably present in an amount of more than 5% by weight, based on the total weight of the composition. Further, it is to be understood that in case water is present in the sunscreen or daily care composition, the sunscreen or daily care composition can be an oil in water emulsion (O/W emulsion), a water in oil emulsion (W/O emulsion), a gel cream or an oil in gel.

In one embodiment of the present invention, the sunscreen or daily care composition can be provided in different forms, e.g. gels, creams, oils, lotions, sticks, or in the form of a sprayable product.

It is particularly preferred that the inventive sunscreen or daily care composition is free of nano UV filter. The term “free of” herein means that the sunscreen or person-care composition comprises less than 1 wt. %, preferably less than 0.5 wt. % and most preferred less than 0.1 wt. % of nano UV filter, each based on the total weight of the sunscreen or daily care composition.

It is even more preferred that the inventive the sunscreen or daily care composition is free of nano material. The term “free of” herein means that the sunscreen or person-care composition comprises less than 1 wt. %, preferably less than 0.5 wt. % and most preferred less than 0.1 wt. % of nano material, each based on the total weight of the sunscreen or daily care composition.

The present invention is further illustrated by the following examples.

### Examples

Two different sunscreen compositions were prepared. The first composition called comparative composition CC1 (CC1) comprises nano-sized 2,2′-methylenebis[6-(2H-1,2,3-benzotriazol-2-yl)-4-(2,4,4-trimethylpentan-2-yl)phenol] (nano MBBT). The second composition called inventive composition 1 (IC1) comprises non-nano MBBT.

Nano MBBT for use in composition CC1 was prepared on lab scale in accordance with example A1, dispersion 1 of WO 2017/198806. 0.51 kg Polyglyceryl-10 Laurate (Dermofeel® G10L, Evonik Industries AG) was dissolved in 2.49 kg deionized water. 3.00 kg 2,2′-methylenebis[6-(2H-1,2,3-benzotriazol-2-yl)-4-(2,4,4-trimethylpentan-2-yl)phenol] (MBBT) was slowly added to this solution. The slurry was pre-grinded for 40 minutes with a stainless-steel rotor mill (MZ 80, FrymaKoruma, ProXES GmbH) operated in circulation mode at 2000 kg/h flow rate and 2900 rpm rotation speed. After pre-grinding, the dispersion was stirred overnight at 70 rpm. 2 kg of the dispersion was milled in a stirred ball mill (Dyno®-Mill Multilab, Willy A. Bachofen AG) equipped with a 0.6 L milling container and 0.3 to 0.4 mm Y—ZrO2 grinding beads (Silibeads®, Sigmund Lindner). The dispersion was circulated through the mill during the grinding process. The particle size of the dispersion was controlled by laser diffraction with a Mastersizer 2000 from Malvern Pananalytical GmbH. The pressure in the mill was below 0.5 bar, the product temperature was below 40° C. Grinding continued until a median particle size Dv50 of about 160 nm was obtained. The product was discharged and active content was controlled.

Non-nano MBBT for use in inventive composition IC1 was prepared using the method described in PCT/EP2022/072536. The slurry contained 50% of UV Filter MBBT, 6.5% of Polyglyceryl-10 Laurate, and water to 100%, The preparation and wet milling of the slurry was performed in the same way as for the Nano MBBT dispersion as described above.

However, the milling in the stirred ball mill was stopped when particle size of the dispersion Dv50 was about 300 nm, and the dispersion was discharged from the mill. 50 g of the dispersion was transferred in a beaker mill equipped with stirrer, and 120 ml of Y—ZrO2 grinding beads (0.6-0.8 mm Y—ZrO2, Silibeads®, Sigmund Lindner) were added. The slurry/beads mixture was homogenized, and then the stirrer speed was increased to 1200 rpm to start the grinding process. Product temperature was set to 45° C. by adjusting the cooling of the beaker mill. 0.3 g of C12-15 Alkylbenzoate (Cetiol® AB, BASF Personal Care & Nutrition GmbH) was added to the suspension and the grinding continued. During the milling, the product temperature increased up to 49-50° C. Particle size was monitored over the entire grinding process, and an increase in Dv10 occurred in parallel with a decrease in Dv90, indicating that nano fraction of particles was reduced during the micronization process. After 4 hours, the stirrer was stopped, the mixture was cooled down, and the suspension was separated from the grinding beads.

The particle size distributions regarding Dv- and D_{N}-values for nano and non-nano MBBT are shown below in Table 1.

D_{N}-values (D_{N}10, D_{N}50, D_{N}90) of nano and non-nano MBBT were determined by using transmission electron microscopy (TEM). For TEM measurements the dispersions were diluted by 1:500 in distilled water. The dispersion was applied on carbon-coated TEM carrier grids by dropping and drying at room temperature. Prior to use, the carbon-coated grids were treated in a plasma-cleaner (Fischione Model 1020; supply gas: ari; Fischione Instruments Inc., PA, USA) to assure a hydrophile surface. The samples were analyzed on a Thermo-Fisher (Waltham, MA, USA) Talos F200i Transmission Electron Microscope in bright-field mode at 200 kV. Images were taken with a low-dose Thermo-Fisher Falcon 3 camera to avoid beam damage of the material. Automated particle size detection from the micrographs was accomplished using the NanoDefine ParticleSizer plugin for ImageJ (FIJI).

Dv-values (Dv10, Dv50, Dv90) of nano and non-nano MBBT were determined by laser diffraction analysis using a Malvern Mastersizer 2000 equipped with a HydroG dispersing unit. Data were analyzed with Malvern standard models suitable for milled samples using optical properties for MBBT taken from the literature (B. Herzog et al, Journal Coll. Int. Sci. 271 (2004) 136-144).

TABLE 1
Particle size distribution of nano MBBT for use in
comparative composition CC1 and non-nano MBBT
for use in inventive composition IC1

| Particle size | Nano MBBT | Non-nano |
|---|---|---|
| distribution | for CC1 | MBBT for IC1 |
| D_{N}10 | 38 nm | 38 nm |
| D_{N}30 | 52 nm | 73 nm |
| D_{N}50 | 69 nm | 143 nm |
| D_{N}90 | 128 nm | 440 nm |
| Dv10 | 77 nm | 159 nm |
| Dv50 | 158 nm | 320 nm |
| Dv90 | 332 nm | 2262 nm |

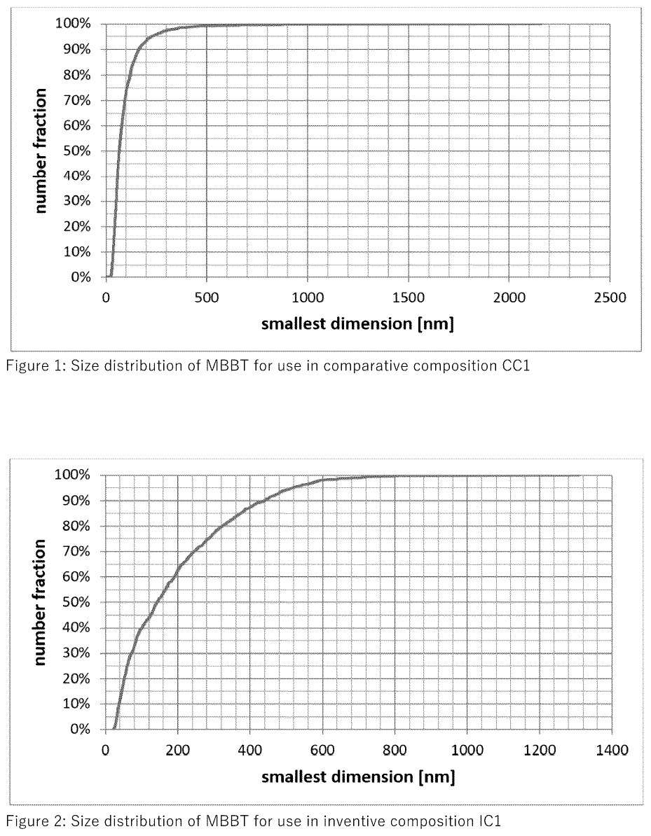

The size distribution of nano MBBT for use in the comparative composition CC1 is also shown in FIG. 1. The size distribution of non-nano MBBT for use in the inventive composition IC1 is also shown in FIG. 2.

Subsequently, sunscreen compositions CC1 and IC1 were prepared with the following components (wt. % based on the total weight of the respective composition):

| | | CC1 | IC1 |
|---|---|---|---|
| part | INCI (trade name) | [wt. %] | [wt. %] |
| A | Sucrose Polystearate and Cetyl | 3.0 | 3.0 |
| | Palmitate (Emulgade ® Sucro Plus) | | |
| | Disodium Cetearyl Sulfosuccinate | 1.0 | 1.0 |
| | (Eumulgin ® Prisma) | | |
| | Cetearyl Alcohol (Lanette ® O) | 1.0 | 1.0 |
| | Dibutyl Adipate (Cetiol ® B) | 10.0 | 10.0 |
| | Propylheptyl Caprylate | 7.0 | 7.0 |
| | (Cetiol ® Sensoft) | | |
| | Diethylamino hydroxybenzoyl hexyl | 5.0 | 5.0 |
| | benzoate, DHHB (Uvinul ® A Plus) | | |
| | Phenoxyethanol and | 1.0 | 1.0 |
| | Ethylhexylglycerin (Euxyl ® PE9010) | | |
| | Ethylhexyl triazone (Uvinul ® T150) | 3.0 | 3.0 |
| B | Water | 59.6 | 58.1 |
| | Glycerine | 2.0 | 2.0 |
| | Xanthan Gum (Rheocare ® XGN) | 0.2 | 0.2 |
| | Disodium EDTA (Edeta ® BD) | 0.2 | 0.2 |
| C | Aqueous suspension of nano MBBT | 5.0 | — |
| | (50 wt. % MBBT active content) with | (2.5 active | |
| | Polyglyceryl-10 Laurate | content) | |
| | Aqueous suspension of non-nano | — | 6.5 |
| | MBBT (38.3 wt. % active content) | | (2.5 active |
| | comprising C12-15 Alkyl Benzoate | | content) |
| | (Cetiol ® AB; 0.6 wt. %) and | | |
| | Polyglyceryl-10 Laurate | | |
| D | Undecane and Tridecane | 2.0 | 2.0 |
| | (Cetiol ® Ultimate) | | |

The compositions CC1 and IC1 were prepared by first preparing parts A and B separately and heating them each to 80° C. under stirring. Under increased stirring, part A was incorporated into part B and homogenized with Ultra Turrax for 10 s at 10000 rpm. The mixture was then allowed to cool to room temperature (298 K) while stirring. Next, part C was added to the mixture and the combined mixture was stirred. Finally, part D was added, and the resulting final mixture was stirred for another 30 minutes at room temperature. The pH value of the mixture was adjusted to be between 6.0 and 6.5.

The absorption spectra of the two compositions were found to be very similar. The in vitro sun protection factor (SPF) for comparative composition CC1 was 20.5 and for inventive composition IC 20.6. Therefore, the two compositions showed equal levels of sun protection. Surprisingly, composition IC1 comprising non-nano MBBT was thus equally effective as sunscreen composition as composition CC1 comprising nano MBBT.

1.-15. (canceled)

16. Sunscreen or daily care composition comprising
i) at least one particulate, micronized organic UV filter having a particle size D_{N}50 of more than 100 nm and a particle size Dv90 of less than 20 μm and
ii) at least one further inorganic or organic UV filter.

17. Sunscreen or daily care composition according to claim 16 characterized in that the at least one particulate, micronized organic UV filter i) has a particle size D_{N}10 of 30 nm or more; and/or
a particle size D_{N}50 of 120 nm or more; and/or
a particle size D_{N}90 of 200 nm or more.

18. Sunscreen or daily care composition according to claim 16 characterized in that the at least one particulate, micronized organic UV filter i) has a particle size Dv10 determined by laser diffraction of more than 0.1 μm; and/or
a particle size Dv50 determined by laser diffraction of less than 0.8 μm;
a particle size Dv90 determined by laser diffraction of less than 10 μm.

19. Sunscreen or daily care composition according to claim 16 characterized in that the at least one particulate, micronized organic UV filter i) is an insoluble organic UV filter.

20. Sunscreen or daily care composition according to claim 19 characterized in that the at least one insoluble organic UV filter is selected from the group consisting of tris-biphenyl triazine, 1,1′-(1,4-piperazinediyl)bis[1-[2-[4-(diethylamino)-2-hydroxybenzoyl]phenyl]-methanone, phenylene bis-diphenyltriazine, and 2,2′-methylenebis[6-(2H-1,2,3-benzotriazol-2-yl)-4-(2,4,4-trimethylpentan-2-yl)phenol].

21. Sunscreen or daily care composition according to claim 16 characterized in that the at least one further inorganic or organic UV filter ii) is selected from the group consisting of
4-(tert.-butyl)-4′-methoxydibenzoylmethane (INCI butyl methoxydibenzoylmethane),
hexyl 2-[4-(diethylamino)-2-hydroxybenzoyl]benzoate (INCI diethylamino hydroxybenzoyl hexyl benzoate),
2,4-bis-{[4-(2-ethyl-hexyloxy)-2-hydroxy]-phenyl}-6-(4-methoxyphenyl)-1,3,5 triazine (INCI bis-ethylhexyloxyphenyl methoxyphenyl triazine),
2-phenyl-1H-benzimidazole-5-sulfonic acid (INCI phenylbenzimidazole sulfonic acid),
[(3Z)-3-[[4-[(Z)-[7,7-dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (INCI terephthalylidene dicamphor sulfonic acid),
disodium phenyl dibenzimidazole tetrasulfonate,
(RS)-2-ethylhexyl-(2E)-3-(4-methoxyphenyl) prop-2-enoate (INCI ethylhexyl methoxycinnamate),
4,4′,4″-(1,3,5-triazin-2,4,6-triyltriimino)tris-benzoic acid tris(2-ethylhexyl) ester (INCI ethylhexyl triazone),
dimethicone diethyl benzalmalonate,
diethylhexyl butamido triazone,
2-ethoxyethyl(2Z)-2-cyano-2-[3-(3-methoxypropylamino)cyclohex-2-en-1-ylidene]acetate,
2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane),
Titanium dioxide,
Zinc oxide,
Cerium oxide, and mixtures thereof.

22. Sunscreen or daily care composition according to claim 21 characterized in that the at least one further inorganic or organic UV filter ii) is an organic UV filter selected from the group consisting of
4-(tert.-butyl)-4′-methoxydibenzoylmethane (INCI butyl methoxydibenzoylmethane)
hexyl 2-[4-(diethylamino)-2-hydroxybenzoyl]benzoate (INCI diethylamino hydroxybenzoyl hexyl benzoate),
2,4-bis-{[4-(2-ethyl-hexyloxy)-2-hydroxy]-phenyl}-6-(4-methoxyphenyl)-1,3,5 triazine (INCI bis-ethylhexyloxyphenyl methoxyphenyl triazine),
4,4′,4″-(1,3,5-triazin-2,4,6-triyltriimino)tris-benzoic acid tris(2-ethylhexyl) ester (INCI ethylhexyl triazone),
2-phenyl-1H-benzimidazole-5-sulfonic acid (INCI phenylbenzimidazole sulfonic acid),
[(3Z)-3-[[4-[(Z)-[7,7-dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (INCI terephthalylidene dicamphor sulfonic acid), and mixtures thereof.

23. Sunscreen or daily care composition according to claim 22 characterized in that the at least one further inorganic or organic UV filter ii) is an organic UV filter selected from the group consisting of
hexyl 2-[4-(diethylamino)-2-hydroxybenzoyl]benzoate (INCI diethylamino hydroxybenzoyl hexyl benzoate),
4,4′,4″-(1,3,5-triazin-2,4,6-triyltriimino)tris-benzoic acid tris(2-ethylhexyl) ester (INCI ethylhexyl triazone), and mixtures thereof.

24. Sunscreen or daily care composition according to claim 16 characterized in that the sunscreen or daily care composition is free of particulate nano UV filter.

25. Sunscreen or daily care composition according to claim 16 characterized in that the sunscreen or daily care composition is free of nano material.

26. Sunscreen or daily care composition according to claim 16 characterized in that the amount of the at least one particulate, micronized organic UV filter i) is from 0.1 to 20 wt. % based on the total weight of the sunscreen or daily care composition.

27. Sunscreen or daily care composition according to claim 16 characterized in that the amount of the at least one further inorganic or organic UV filter ii) is from 0.1 to 20 wt. % based on the total weight of the sunscreen or daily care composition.

28. Sunscreen or daily care composition according to claim 16 characterized in that the at least one particulate, micronized organic UV filter i) is provided in form of an aqueous suspension.

29. Sunscreen or daily care composition according to claim 28 characterized in that the aqueous suspension comprises a dispersing agent, in particular wherein the dispersing agent is selected from the group consisting of polyglycerol alkyl ester and alkyl polyglucoside.

30. Sunscreen or daily care composition according to claim 28 characterized in that the aqueous suspension comprises a hydrophobic additive, in particular wherein the hydrophobic additive is selected from the group consisting of alcohols having from 6 to 18 carbon atoms; C6-C24 carboxylic acids; esters of C6-C24 carboxylic acids with C3-C24 alcohols; esters of hydroxycarboxylic acids with C6-C24 alcohols; esters of carboxylic acids with polyhydric alcohols; liquid mono-/di-/tri-glyceride mixtures based on C6-C18 carboxylic acids; esters of C6-C24 alcohols with aromatic carboxylic acids or with oxo carboxylic acids; tricarboxylic acid esters; esters of C2-C12 dicarboxylic acids with alcohols having from 1 to 22 carbon atoms or polyols having from 2 to 10 carbon atoms and from 2 to 6 hydroxy groups; substituted cyclohexanes; C6-C22 alcohol carbonates; symmetric or asymmetric dialkyl ethers having a total of from 12 to 36 carbon atoms; ring-opening products of epoxidized carboxylic acid esters with polyols; silicone oils; aliphatic or naphthenic hydrocarbons; diol esters; and mixtures thereof.
